(19) United States
(12) Patent Application Publication
(10) Pub. No.: US 2008/0293621 A1
(43) Pub. Date: Nov. 27, 2008

(54) Methods for Prolonging Feline Life

(76) Inventors: Timothy Arthur Allen, Lawrence, KS (US); Kimberly Lynette Scott, Topeka, KS (US); Claudia Ann Kirk, Louisville, TN (US); Christopher Sidney Cowell, Chesterfield, MO (US)

Correspondence Address: COLGATE-PALMOLIVE COMPANY, 909 RIVER ROAD, PISCATAWAY, NJ, 08855, US

(21) Appl. No.: 11/912,637

(22) PCT Filed: Apr. 28, 2006
(86) PCT No.: PCT/US06/16326

§ 371 Date: Jun. 18, 2008

Related U.S. Application Data
(60) Provisional application No. 60/676,624, filed on Apr. 29, 2005.
(60) Provisional application No. 60/692,780, filed on Jun. 22, 2005.

(51) Int. Cl. A61K 38/22; A61K 31/593; A61P 13/12; A23K 1/18

(52) U.S. Cl. 514/8; 514/167; 426/2

(57) ABSTRACT

Methods for prolonging feline life by maintaining a feline on a food having reduced levels of protein, phosphorus, and sodium when compared to a standard feline maintenance food.

This application claims priority to U.S. Provisional Application Ser. No. 60/676,624, filed Apr. 29, 2005, and U.S. Provisional Application Ser. No. 60/692,780, filed Jun. 22, 2005, the disclosures of which are incorporated herein by reference.

### BACKGROUND OF THE INVENTION

1. Field of the Invention

The present invention relates generally to methods for prolonging feline life and particularly to the use of foods having reduced levels of protein, phosphorus, and sodium to prolong feline life.

2. Description of the Prior Art

Chronic renal failure (CRF) is common in middle-aged to older felines and the frequency of occurrence of CRF appears to be increasing. It is estimated that, in 2000, the prevalence of renal failure among cats aged 10 years or older was about 27% and among cats aged 15 years or older as high as about 49%.

Most animals experience a progressive decline in renal function over their life span. Felines, unlike most animals, do not experience a significant decline in renal function over their life span but instead experience a rapid onset of the signs of CFR at some point in later life. CRF is characterized by progressive structural lesions resulting in impairment of renal excretory, biosynthetic, and regulatory functions.

Dietary modification is a mainstay of therapy to minimize external manifestations of spontaneous CRF in felines. However, no randomized, masked, controlled clinical trials have apparently been reported to evaluate long-term effectiveness of “renal foods”, i.e., dietary regimens adapted for felines with naturally occurring renal failure, and in particular the effect of such foods on longevity of felines. There is, therefore, a need for new methods for management of feline renal failure and disease and therefore prolonging the life of felines.

### SUMMARY OF THE INVENTION

It is, therefore, an object of the present invention to provide methods for prolonging feline life.

It is another object of the invention to provide methods for delaying the onset of feline renal failure.

It is a further object of the invention to decrease the morbidity and mortality caused by feline renal disease.

It is another object of the invention to provide articles of manufacture in the form of kits that contain combinations of compositions and devices useful for prolonging feline life, delaying the onset of feline renal failure, and decreasing the morbidity and mortality caused by feline renal disease.

These and other objects are achieved using novel methods for prolonging the life of felines, delaying the onset of feline renal failure, and decreasing the morbidity and mortality caused by feline renal disease. The methods comprise maintaining a feline on a food having reduced levels of protein, phosphorus, and sodium when compared to a standard feline food.

Other and further objects, features and advantages of the present invention will be readily apparent to those skilled in the art.

### DETAILED DESCRIPTION OF THE INVENTION

### Definitions

The term “prolonging the life” means increasing the chronological age of an animal at its death by natural causes or by euthanasia when, in the judgment of the animal's caregiver or a veterinarian, quality of life for the animal has seriously and irreversibly deteriorated.

The term “feline” means an animal of any feline species, including domestic cats (Felis domesticus) and their wild and feral conspecific relatives, and further including without restriction wild, exotic and captive animals of other feline species such as lions, tigers, panthers, etc., including those kept for scientific, educational or entertainment purposes. Preferably, the feline is a pet cat.

The term “substantially the full nutritional requirement for maintenance” means at least a maintenance amount of the macronutrients (protein, carbohydrate, lipid and fiber) required by a feline, according to guidelines published and from time to time updated by bodies such as NRC (National Research Council) and AAFCO (Association of American Feed Control Officials), subject to the reduced protein level of the food as defined herein. Optionally, the food further comprises at least a maintenance amount of one or more micronutrients, such as vitamins, essential amino acids, essential fatty acids and minerals, subject to the reduced phosphorus and sodium levels of the food as defined herein, although micronutrients can additionally or alternatively be supplied in the form of one or more supplements not forming part of the one or more food compositions making up the food. Further information on nutritional requirements for maintenance of adult felines can be found, for example, in sources such as NRC (2003) Nutrient Requirements of Dogs and Cats, pp. 431-434.

The term “maintaining a feline on a food” means providing to a feline for consumption a regular amount, e.g., a daily amount, of one or more food compositions that together satisfy substantially the full nutritional requirement for maintenance of the feline. The food is not necessarily constant during the period of such feeding so long as substantially the full nutritional requirement for maintenance of the animal is satisfied and the food has reduced levels of protein, phosphorus, and sodium as defined herein. For example, the feline can be switched as often as desired from one brand of cat food to another, or from a wet composition (e.g., a canned cat food) to a dry composition (e.g., a kibble), or vice versa.

The term “renal drug” means any compound, composition, or drug useful for preventing or treating renal failure or kidney disease.

This invention is not limited to the particular methodology, protocols, and reagents described herein because they may vary. Further, the terminology used herein is for the purpose of describing particular embodiments only and is not intended to limit the scope of the present invention. As used herein and in the appended claims, the singular forms “a,” “an,” and “the” include plural reference unless the context clearly dictates otherwise, e.g., reference to “a host cell” includes a plurality of such host cells.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the preferred methods, devices, and materials are described herein.

All patents, patent application, and publications mentioned herein are incorporated herein by reference to the extent allowed by law for the purpose of describing and disclosing the compounds and methodologies reported therein that might be used with the present invention. However, nothing herein is to be construed as an admission that the invention is not entitled to antedate such disclosure by virtue of prior invention.

### The Invention

In various aspects, the present invention provides methods for (1) prolonging feline life, (2) delaying the onset of feline renal failure, and (3) decreasing the morbidity and mortality caused by feline renal disease. The methods comprise maintaining a feline on a food having reduced levels of protein, phosphorus, and sodium when compared to a standard feline maintenance food. The invention is based upon the novel discovery that feline kidney function can be altered by maintaining a feline on a food relatively low in protein, phosphorus, and sodium and that altering kidney function can prolong feline life, delay the onset of feline renal failure, and decrease the morbidity and mortality of feline kidney disease.

A “standard feline maintenance food” is a food meeting the nutritional requirements for maintenance of a healthy adult feline, e.g., as set forth by NRC or AAFCO in the herein-cited references, without being “high” in any one of protein, phosphorus, or sodium. A feline food “high” in protein is one having a protein content greater than about 50% of dry matter and/or providing more than about 10 g protein per 100 kcal ME. A feline food “high” in phosphorus is one having a phosphorus content greater than about 1.2% of dry matter and/or providing more than about 0.25 g phosphorus per 100 kcal ME. A feline food “high” in sodium is one having a sodium content greater than about 0.5% of dry matter and/or providing more than about 0.1 g sodium per 100 kcal ME. An illustrative standard feline maintenance food that is not “high” in protein, phosphorus, or sodium comprises, on a dry matter basis, about 45% protein, about 1% phosphorus and about 0.4% sodium, and provides about 9 g protein, about 0.2 g phosphorus and about 0.08 g sodium per 100 kcal ME.

A feline food having “reduced levels” of protein, phosphorus, and sodium is one having sufficiently lower levels than those in a standard feline maintenance food such that a feline consuming the food experiences a prolonged life, a delay the onset of renal failure, and a decrease in the morbidity and mortality associated with kidney disease. Typically, to achieve the benefits of the present invention, levels of protein, phosphorus, and sodium in the food are reduced by at least about 10% from those in a standard feline maintenance food.

In one embodiment wherein each of phosphorus and sodium in the food are reduced by at least about 10% when compared to a standard feline maintenance food, protein is reduced by from about 12% to about 60%, preferably from about 18% to about 48%, from about 24% to about 44%, or from about 30% to about 40%. Protein content of the food, on a dry matter basis, according to this embodiment is from about 18% to about 40%, preferably from about 23% to about 37%, from about 25% to about 34% or from about 27% to about 32%, illustratively from about 28% to about 30%. Expressed on an ME basis, protein content of the food should be from about 3.6 to about 7.9 g/100 kcal ME, preferably from about 4.7 to about 7.4, from about 5.0 to about 6.8 or from about 5.4 to about 6.3 g/100 kcal ME.

In another embodiment wherein each of protein and sodium in the food are reduced by at least about 10% when compared to a standard feline maintenance food, phosphorus is reduced by from about 15% to about 80%, preferably from about 20% to about 75%, from about 30% to about 65%, from about 40% to about 60%, or from about 40% to about 55%. Phosphorus content of the food, on a dry matter basis, according to this embodiment is from about 0.2% to about 0.85%, preferably from about 0.25% to about 0.8%, from about 0.35% to about 0.7%, from about 0.4% to about 0.6%, or from about 0.45% to about 0.6%. Expressed on an ME basis, phosphorus content of the food should be from about 0.04 to about 0.17 g/100 kcal ME, preferably from about 0.05 to about 0.16, from about 0.07 to about 0.14, from about 0.08 to about 0.12, or from about 0.09 to about 0.12 g/100 kcal ME.

In another embodiment wherein each of protein and phosphorus in the food are reduced by at least about 10% when compared to a standard feline maintenance food, sodium is reduced by from about 12% to about 90%, preferably from about 15% to about 80%, from about 18% to about 70% or from about 21% to about 60%. Sodium content of the food, on a dry matter basis, according to this embodiment is from about 0.04% to about 0.35%, preferably from about 0.08% to about 0.34%, from about 0.12% to about 0.33% or from about 0.16% to about 0.32%. In one embodiment sodium content is from about 0.2% to about 0.35%, on a dry matter basis. Expressed on an ME basis, sodium content of the food should be from about 0.008 to about 0.07 g/100 kcal ME, preferably from about 0.016 to about 0.07, from about 0.02 to about 0.07 or from about 0.03 to about 0.06 g/100 kcal ME.

Illustrative canned feline foods wherein each of protein, phosphorus, and sodium are reduced as defined herein include, without limitation, Hill's® Feline k/d®, Hill's Feline k/d® with Chicken, Hill's® Feline g/d®, Hill's Feline l/d®, Purina Veterinary Diet™ NF and Royal Canin Veterinary Diet™ Renal LP 21™. For example, Hill's® Feline k/d® canned food is stated to contain (average nutrient contents): protein: 8.4% as fed, 28.6% dry matter, 6.6 g/100 kcal; phosphorus: 0.10% as fed, 0.34% dry matter, 0.078 g/100 kcal; sodium: 0.09% as fed, 0.31% dry matter, 0.07 g/100 kcal.

Illustrative dry feline foods wherein each of protein, phosphorus, and sodium are reduced as defined herein include, without limitation, Hill's® Feline k/d®, Hill's® Feline g/d®, Hill's® Feline l/d®, Hill's® Science Diet® Adult Ocean Fish & Rice Recipe, Purina Veterinary Diet™ NF and Royal Canin Veterinary Diet™ Renal LP 21™. For example, Hill's® Feline k/d® dry food is stated to contain (average nutrient contents): protein: 26.2% as fed, 28.3% dry matter, 6.7 g/100 kcal; phosphorus: 0.45% as fed, 0.49% dry matter, 0.114 g/100 cal; sodium: 0.23% as fed, 0.25% dry matter, 0.058 g/100 kcal.

The maintenance of a feline on a food of the present invention is a long-term endeavor. For example, the feline can be maintained on a food according to the present invention for a period of at least about 6 months, at least about 1 year, at least about 2 years, at least about 3 years, or for a period beginning after onset or initial diagnosis of the renal failure or disease and continuing for substantially the remainder of the feline's life. Given the long-term nature of the present methods, it will be understood that it is generally undesirable to restrict any one of protein, phosphorus, and sodium to a level that is below a minimum consistent with overall feline health. According to one embodiment, therefore, protein is not reduced below about 25% dry matter or about 5 g/100 kcal ME. According to another embodiment, phosphorus is not reduced below about 0.45% dry matter or about 0.09 g/100 kcal ME. According to another embodiment, sodium is not reduced below about 0.04% dry matter or about 0.008 g/100 kcal ME.

In one embodiment, the food contains from about 27% to about 32% protein, from about 0.36% to about 0.54% phosphorus, and from about 0.15% to about 0.32% sodium, on a dry matter basis. Such a food typically provides from about 5.4 to about 6.3 g protein, from about 0.08 to about 0.12 g phosphorus, and from about 0.03 to about 0.06 g sodium per 100 kcal ME. Maintenance of felines having a renal disease on such a ration exemplifies the present invention by prolonging life in comparison with a standard feline maintenance food containing about 45% protein, about 0.9% phosphorus, and about 0.4% sodium, that provides about 9 g protein, about 0.2 g phosphorus, and about 0.08 g sodium per 100 kcal ME. The life-prolonging effect of a food as described herein is particularly surprising when it is recognized that the standard food used as a comparator is itself not “high” as defined above in any one of protein, phosphorus or sodium.

In another embodiment, the food comprises a dry food comprising protein in an amount of from about 5% to about 40%, phosphorus in an amount of from about 0.01% to about 2%, and sodium in an amount of from about 0.01% to about 2% on an “as fed” basis. According to this embodiment, on an “as fed” basis, the protein content of the dry food can be from about 10% to about 30%, or from about 24% to about 30%; the phosphorus content can be from about 0.05% to about 1%, or from about 0.2% to about 0.5%; and/or the sodium content can be from about 0.05% to about 1%, or from about 0.15% to about 0.35%.

In another embodiment, the food comprises a moist food comprising protein in an amount of from about 4% to about 12%, phosphorus in an amount of from about 0.03% to about 0.2%, and sodium in an amount of from about 0.03% to about 0.2% on an “as fed” basis. According to this embodiment, on an “as fed” basis, the protein content of the moist food can be from about 5% to about 11%, or from about 6% to about 9%; the phosphorus content can be from about 0.05% to about 0.15%, or from about 0.08% to about 0.1%; and/or the sodium content can be from about 0.05% to about 0.15%, or from about 0.08% to about 0.1%.

Optionally, the food can be modified for nutrients other than protein, phosphorus, and sodium. For example, the food can be supplemented with polyunsaturated fatty acids, more particularly omega-3 fatty acids such as docosahexaenoic acid (DHA) and/or eicosapentaenoic acid (EPA). Many other such modifications are known to skilled artisans.

Dietary allowances may be expressed in relation to metabolizable energy (ME) content of the food, for example in g/MJ or g/100 kcal. Protein herein is expressed as total crude protein unless otherwise indicated. Percentages for compositions herein are expressed on a “dry matter” basis unless specifically stated otherwise.

A further method comprises maintaining a feline on a food having reduced levels of protein, phosphorus, and sodium when compared to a standard feline food while administering one or more renal drugs to the feline for a prescribed period. Typically, health care professionals, e.g., doctors and veterinarians, diagnose kidney disease in a feline and prescribe a renal drug (any drug useful to prevent or treat kidney disease in a feline) to treat the disease. The feline is administered the renal drug until the symptoms cease and the disease is considered cured or administration is continued indefinitely for chronic kidney disease. In the present invention, the feline is maintained on the food having reduced levels of protein, phosphorus, and sodium and renal drugs are administered to the feline for treatment of the disease. Renal drugs useful in the invention are any renal drugs known to skilled artisans to be useful for combating kidney disease. Preferred drugs include lysosome-activating compounds such as those described in U.S. Pat. No. 6,589,748, triterpene saponins such as those described in U.S. Pat. No. 6,784,159, activin inhibitors such as those described in U.S. Pat. No. 6,599,876 and US Patent Application Number (USPAN) 20020028762, integrin receptor inhibitors and TGF inhibitors such as those described in U.S. Pat. No. 6,492,325, TGF activation inhibitors such as those described in U.S. Pat. No. 6,458,767, and insulin-like growth factor (IGF) as described in U.S. Pat. No. 5,723,441. Most Preferred drugs include Converting Enzyme (ACE) inhibitors, androgens, erythropoiten, and calcitriol. Angiotensin and endothelin are potent systemic vasoconstrictors with specific intrarenal effects that contribute to progressive renal injury. A variety of renal drugs are used to mitigate the effect of these vasoconstrictors. Angiotensin converting enzyme inhibitors (enalapril—Enacard and Vasotec and benazepril—Lotensin) have been associated with a reduction in the severity of proteinuria and slowing of progression of renal failure. The ACE inhibitor enalapril (Enacard, Vasotec) limits glomerular and systemic hypertension, proteinuria, and glomerular and tubulointerstitial lesions. Angiotensin blockers and endothelin inhibitors have beneficial effects in renal disease. Vasopeptide inhibitors are agents that inhibit both ACE and neutral endopeptidase, an enzyme involved in the breakdown of natriuretic peptides, adrenomedullin, and bradykinin. These renal drugs decrease angiotenin II production and increase accumulation of vasodilators. Renal patients with systemic hypertension respond to calcium channel blockers such as amlodipine (Norvasc). Uremic gastritis (esophagitis, gastritis, gastric ulceration and hemorrhage) is treated with H2 receptor antagonists (cimetidine—Tagamet, famotidine—Pepcid), proton pump blockers (omeprazole—Prilosec), cytoprotective agents (misoprostol—Cytotec), and antiemetic drugs that effect the emetic center (chlorpromazine—Thorazine, perchlorperazine—Compazine, metoclopramide—Reglan). Androgens or anabolic steroids (Stanozol, Winstrol-V) are used in the treatment of anemia associated with chronic renal failure. Hormone replacement therapy using recombinant human (or other species) erythropoiten (Epoetin alpha, Epogen, Procrit) is the treatment of choice for severe anemia associated with renal failure. Phosphate binders (aluminum hydroxide—Amphojel, aluminum carbonate—Basaljel) are used to control hyperphosphatemia and secondary renal hyperparathyroidism. Calcitriol (1, 25-dihydroxycholecalciferol) (Rocaltrol) and vitamin D analogues cause a calcium-independent suppression of parathyroid hormone (PTH). Administration of phosphate binders, calcitriol and related compounds has been advocated in chronic renal failure to prevent multi-system toxicity caused by PTH. Potassium depletion and hypokalemia are common in cats with chronic renal failure. Oral supplementation of potassium in the form of potassium gluconate or citrate is recommended. Holistic renal drugs and compositions are also included in the present invention. Preferred holistic renal drugs include cranberry extract and mannose. Cranberry extract is purported to reduce the prevalence of urinary tract infection which is a common risk factor for long-term decline of renal function. Renal drugs include typical small molecule pharmaceuticals, small proteins, macromolecular proteins and molecules, and antibodies and further include vaccines designed to prevent renal disease. Antibodies include polyclonal and monoclonal antibodies and immunoglobulin fragments such as Fv, Fab, Fab′, F(ab′)2, or other antigen-binding antibody subsequences that interact with an antigen and perform the same biological function as a native antibody. The renal drugs are administered to the feline using any method appropriate for the renal drug and in amounts known to skilled artisans to be sufficient to treat or prevent renal disease.

In a further aspect, the present invention provides a kit useful for prolonging feline life, delaying the onset of feline renal failure, and decreasing the morbidity and mortality caused by feline renal disease comprising in separate containers in a single package a food composition suitable maintaining a feline on a food having reduced levels of protein, phosphorus, and sodium when compared to a standard feline food and one or more renal drugs.

In other embodiments, the kits further comprise one or more renal diagnostic devices for determining kidney function and evaluating the presence and severity of kidney disease in a feline in a separate package. The renal diagnostic devices useful in the present invention include any device suitable for determining kidney function and evaluating the presence and severity of kidney disease in a feline. Preferred diagnostic methods include serum urea nitrogen (SUN), creatinine levels, urine specific gravity, and DNA damage, including urine assays for albumin such as those described in U.S. Pat. No. 6,589,748, U.S. Pat. No. 6,447,989 and USPAN 20050026225 and comet trail assays. Diagnostic methods are based upon known techniques including (1) blood markers such as elevated blood urea nitrogen concentration, elevated serum creatinine concentration, hyperphosphatemia, hyperkalemia or hypokalemia, metabolic acidosis and hypoalbuminemia, (2) urine markers such as impaired urine concentrating ability, proteinuria, cylinduria, renal hematuria, inappropriate urine pH, inappropriate urine glucose concentration, and cystinuria, (3) physical, imaging, and diagnostic markers such as size, shape, location, and density, (4) single nucleotide polymorphisms (SNPs) such as those disclosed in WO 2004113570 A2, (5) genetic profiles that are indicative of kidney disease, (6) proteomic profiles that are indicative of kidney disease, and (7) metabolic profiles that are indicative of kidney disease. These diagnostic methods and devices (e.g., test strips, ELISA assays, comet assays,) based upon such methods are commonly available to skilled artisans such as scientists and health care professionals and many are available to consumers, e.g., the Heska Corporation's (Fort Collins Colorado) E.R.D.-HealthScreen Urine Tests that detects small amounts of albumin in the urine (“microalbuminuria”).

In one embodiment, a periodic diagnostic assessment for the presence or progress of renal disease is conducted. Based upon the results of such diagnostic assessment, the food and/or renal drug dosage or regimen can be adjusted. Diagnostic assessment can be based on one or more criteria independently selected from blood markers (e.g., elevated serum urea nitrogen concentration, elevated serum creatinine concentration, hyperphosphatemia, hyperkalemia, hypokalemia, metabolic acidosis, hypoalbuminemia and the like), urine markers (e.g., impaired urine concentrating ability, proteinuria, cylinduria, renal hematuria, inappropriate urine pH, inappropriate urine glucose concentration, cystinuria and the like), physical observations and measurements, imaging, SNPs, genetic profiles, proteomic profiles and metabolic profiles and can be determined using one or more renal diagnostic devices.

In a further aspect, the present invention provides a kit useful for prolonging feline life, delaying the onset of feline renal failure, and decreasing the morbidity and mortality caused by feline renal disease comprising in separate containers in a single package a food composition suitable maintaining a feline on a food having reduced levels of protein, phosphorus, and sodium when compared to a standard feline food and one or more renal diagnostic devices.

The kits further comprise information about or instructions for using the methods and kits of the present invention to prolong feline life, delay the onset of feline renal failure, or decrease the morbidity and mortality caused by feline renal disease.

In another aspect, the present invention provides a means for communicating information about or instructions for using foods having reduced levels of protein, phosphorus, and sodium when compared to a standard feline maintenance food to prolong feline life, delay the onset of feline renal failure, and decrease the morbidity and mortality caused by feline renal disease. In another embodiment, the invention further provides information about or instructions for using the foods in combination with renal drugs to prevent or treat kidney disease. In another, the invention further provides information about or instructions for using the renal diagnostic devices of the present invention. The communicating means comprises a document, digital storage media, optical storage media, audio presentation, or visual display containing the information or instructions. Preferably, the communication is a displayed web site or a brochure, product label, package insert, advertisement, or visual display containing such information or instructions. Useful information includes one or more of (1) methods and techniques for combining the food and the renal drugs, (2) information for using the renal diagnostic devices, (3) details about the side effects, if any, caused by using the present invention in combination with other drugs, and (4) contact information for feline caregivers to use if they have a question about the invention and its use. Useful instructions include dosages, administration amounts and frequency, and administration routes. The communication means is useful for instructing a feline caregiver on the benefits of using the present invention.

The methods and kits are useful for decreasing the morbidity and mortality for felines susceptible to or suffering from renal failure or kidney disease and therefore prolonging the life of the feline.

### EXAMPLES

This invention can be further illustrated by the following examples of preferred embodiments thereof, although it will be understood that these examples are included merely for purposes of illustration and are not intended to limit the scope of the invention unless otherwise specifically indicated.

### Example 1

A study was conducted to compare a renal food (modified in protein, phosphorus, and sodium composition) with an adult maintenance food in minimizing uremic episodes and mortality rate in cats with mild to moderate spontaneous CRF. To examine impact of dietary therapy on development of uremic crisis, mortality rate and progression of renal failure, a combination of dietary modifications commonly recommended to manage CRF was incorporated. The design of this type of study is likely to provide a more informative and efficient evaluation than a series of clinical trials in which the therapeutic efficacies of individual dietary components are studied singly. In addition, studying a combination of dietary modifications encompasses evaluations of the overall interactions of various dietary components. The term “food” in this Example means a food as fed to adult felines and does not necessarily represent the totality of food consumed by the felines.

### Material and Methods

Cat Selection. Forty-five cats were recruited from the Minneapolis-St. Paul area by mailing a description of the study to area veterinarians asking for referrals. Cats were also recruited from the University of Minnesota Veterinary Medical Center patient population using a medical records search and direct owner contact. Cats were considered for enrollment if they were greater than one year of age and had a history consistent with kidney disease of at least four weeks duration. During the first screening visit, patients were evaluated with a defined medical history, physical examination, indirect blood pressure measurement and measurement of serum creatinine concentration. If the serum creatinine concentration was 2.1 to 4.5 mg/dl and there was no evidence of pre-renal azotemia, patients were re-evaluated 7 to 21 days later. At that time, if the serum creatinine concentration did not increase or decrease by >20% from the initial value and remained 2.1 to 4.5 mg/dl, the patient was provisionally enrolled for study.

After provisional acceptance, the cats were re-evaluated on the basis of results of a medical history, physical examination, complete blood count, serum chemistry profile, urinalysis, indirect blood pressure determination, survey abdominal radiography, serum T4 concentration and ELISA tests for Feline Leukemia Virus (FeLV) and Feline Immunodeficiency Virus (FIV). Cats meeting all inclusion and exclusion criteria, as listed in Table 1, were then fed a combination food as set forth in Table 2 for 6 weeks.

TABLE 1
Inclusion and Exclusion Criteria

| Inclusion Criteria | Exclusion Criteria |
|---|---|
| Cats at least 1 year of age | Uremic signs (anorexia, vomiting, lethargy) |
| Serum creatinine concentration 2.1-4.5 mg/dl | Cats with renal disease requiring treatment with: |
| Stable intrarenal azotemia defined as serum | Antinausea drugs |
| creatinine concentration within 20% of | Anti-hypertensive drugs |
| baseline value over 7-21 days. | Parenteral fluid supplementation |
| | Vitamin supplementation |
| | Phosphate binders |
| | Alkalinizing agents |
| | Potassium supplements |
| | Recombinant human erythropoietin |
| | Current ureteroliths |
| | Treatment with corticosteroids |
| | Expected to die of non-renal illnesses (neoplasia, |
| | congestive heart failure, diabetes mellitus, FeLV, |
| | FIV) |

TABLE 2
Composition of Combination Diet

| Nutrient | Dry | Moist |
|---|---|---|
| Protein (% DM) | 36 | 41 |
| Crude Fiber (% DM) | 0.8 | 2.4 |
| Crude Fat (% DM) | 25.8 | 26.0 |
| ME (kcal/g) | 4.3 | 1.3 |
| Calcium (% DM) | 0.93 | 0.8 |
| Phosphorus (% DM) | 0.7 | 0.7 |
| Sodium (% DM) | 0.5 | 0.4 |
| Potassium (% DM) | 1.0 | 0.9 |
| Crude fiber (% DM) | 0.8 | 2.4 |
| Omega-6 fatty acids (% DM) | 3.7 | 3.2 |
| Omega-3 fatty acids (% DM) | 0.21 | 0.17 |
| Moisture (%) | 5.4 | 70.8 |

At the end of 6 weeks, 45 cats of 7 breeds (domestic short hair 30, domestic long hair 7, Siamese 4, Maine Coon 1, Birman 1, Himalayan 1) and ranging in weight from 2.47 to 7.65 kg (mean 4.27 (1.08 kg)) met all inclusion and exclusion criteria. They were randomly assigned to either a renal food or a maintenance food.

Foods. This study was designed to compare a typical adult feline dry and moist maintenance food with a dry and moist food designed specifically for treatment of feline chronic kidney disease (renal food), as shown in Table 3.

TABLE 3
Composition of Renal and Maintenance Foods

| | Dry | | Moist | |
|---|---|---|---|---|
| Nutrient | Renal | Maintenance | Renal | Maintenance |
| Protein (% DM) [% ME] | 28 [20] | 45 [36] | 28 [31] | 46 [39] |
| NFE (% DM) [% ME] | 44 [33] | 22 [12] | 38 [18] | 20 [14] |
| Fat (% DM) [% ME] | 22 [47] | 26 [51] | 27 [51] | 26 [46] |
| ME (kcal/g) | 4.3 | 4.7 | 1.2 | 1.3 |
| Calcium (% DM) | 0.7 | 1.1 | 0.6 | 0.9 |
| Phosphorus (% DM) | 0.5 | 0.9 | 0.5 | 1.0 |
| Sodium (% DM) | 0.2 | 0.4 | 0.3 | 0.4 |
| Potassium (% DM) | 0.7 | 0.9 | 1.1 | 0.9 |
| Crude fiber (% DM) | 0.8 | 0.7 | 1.7 | 3.4 |
| Omega-6 fatty acids | 3.1 | 3.9 | 4.6 | 3.0 |
| (% DM) | | | | |
| Omega-3 fatty acids | 0.2 | 0.2 | 0.6 | 0.2 |
| (% DM) | | | | |
| Moisture (%) | 6.4 | 5.5 | 70.7 | 73.1 |

DM = dry matter.
ME = Metabolizable energy (AAFCO tested)
NFE = Nitrogen free extract; represents carbohydrate fraction of the food

Both foods provided complete and balanced nutrition for the maintenance of adult cats as substantiated by AAFCO feeding trials (renal food) or by exceeding the minimum AAFCO nutrient profile for an adult cat (maintenance food). The digestibility of each food was based on ingredient digestibility testing. Protein digestibility was 78.8% (dry) and 77.3% (moist) for the renal food and 83.4% (dry) and 82.5% (moist) for the maintenance food. Principal characteristics of the renal food were reduced quantities of protein, phosphorus, and sodium compared to the maintenance food. The renal food was supplemented with omega-3 polyunsaturated fatty acids, whereas the maintenance food was not.

Feeding Protocol. After qualifying for the study, all cats were acclimated over 6 weeks to a combination food (Table 2) formulated to represent the nutritional average of the renal food and maintenance food. The goal was to gradually decrease consumption of the amount of regular food while increasing the amount of the combination food so that cats would be consuming at least 80% of the combination food for 3 weeks prior to random assignment to the study foods. Cats were fed this combination food to minimize variability associated with consumption of different foods fed by owners before being enrolled in the study. This strategy was also chosen to minimize abrupt changes in dietary ingredients at the time of randomization to the renal food or maintenance food group. Throughout the study, owners were asked to continue the method of feeding (free-choice or meal fed) used prior to entry into the study. Owners were instructed to give a quantity of food sufficient to maintain adequate nutrition based on serial assessments of body weight and physical examination. Free-choice feeding was limited to a predetermined amount of food based on daily caloric requirement as determined by the formula 1.5 (30×BW (kg)+70)=CR (kcal/day), where BW=body weight and CR=caloric requirement.

Owners were asked to monitor the quantity of study food consumed and to note the amount and type of any other foods consumed, using a printed record. When body weight decreased, the owners were asked to increase the amount of food given to the patient. In some cats, food intake was enhanced by warming the food, or by the use of flavoring agents (diluted chicken broth, diluted tuna water, etc.).

Study Design. A randomized, double-masked, controlled clinical trial was performed. Owners of cats that met all inclusion and exclusion criteria were asked to review and sign an informed consent form. Then, over 6 weeks, all cats were acclimated to the combination food. After 6 weeks and immediately prior to random assignment to the maintenance food or renal food, each cat was evaluated by use of a defined medical history, physical examination, body condition score, expanded serum biochemistry panel (i.e., serum urea nitrogen, creatinine, glucose, inorganic phosphorus, calcium, sodium, potassium, chloride, total CO2, albumin, total bilirubin and total protein concentrations, and serum amylase, alanine transaminase and alkaline phosphatase activities), complete blood count, venous blood gas, urinalysis, quantitative urine culture for aerobic bacteria, indirect blood pressure measurements, serum ionized calcium and serum parathyroid hormone concentration, as set forth in Table 4. Although all of these analytes were evaluated throughout the clinical trial, only analytes related to renal failure (serum creatinine, urea nitrogen, phosphorous, total calcium, ionized calcium, parathyroid hormone, total CO₂, and albumin concentrations) were compared between the two food groups.

TABLE 4
Schedule of Diagnostic Procedures During Study

| | Months from date of assignment to test food | | | | | | | | | | | |
|---|---|---|---|---|---|---|---|---|---|---|---|---|
| Procedure | −2 | −1.5 | 0 | 1 | 3 | 6 | 9 | 12 | 15 | 18 | 21 | 24 |
| Medical history | x | x | x | x | x | x | x | x | x | x | x | x |
| Quality of life questionnaires | | x | x | x | x | x | x | x | x | x | x | x |
| Body condition score | x | x | x | x | x | x | x | x | x | x | x | x |
| Physical examination | x | x | x | x | x | x | x | x | x | x | x | x |
| Serum biochemistry panel | | x | x | | x | x | x | x | x | x | x | x |
| Complete blood count | | x | x | | x | x | x | x | x | x | x | x |
| Urinalysis | | x | x | | x | x | x | x | x | x | x | x |
| Quantitative urine culture | | x | x | | x | x | x | x | x | x | x | x |
| Indirect blood pressure | x | x | x | x | x | x | x | x | x | x | x | x |
| UP/UC ratio | | | x | | | x | | x | | x | | x |
| PTH and ionized calcium | | | x | | | x | | x | | x | | x |
| T₄ [T(four)] | | x | | | | | | | | | | |
| FeLV/FIV ELISA | | x | | | | | | | | | | |
| PCV/TPP | | | | x | | | | | | | | |
| SUN/TCO₂/SCr/K⁺ | | | | x | | | | | | | | |
| K⁺/SCr/TCO₂/ALT | x | | | | | | | | | | | |
| Survey abdominal radiography | | x | | | | | | | | | | x |

PTH = parathyroid hormone;
SUN = Serum urea nitrogen;
TCO₂ = Total carbon dioxide;
SCr = Serum creatinine;
PCV = Packed cell volume;
TPP = Total plasma protein

Cats were assigned to either the renal food or the maintenance food using block randomization (blocks of 8) in a ratio of 1 to 1. Assignment was made via sealed, sequentially numbered, envelopes. Double masking of the study was maintained by use of coded, identical packaging material.

One month after assignment to either the renal food or the maintenance food, the status of each cat was evaluated by a history, physical examination, indirect blood pressure, limited serum biochemical profile (serum urea nitrogen, creatinine, potassium and total CO2 concentrations), PCV and total plasma solids concentration determinations.

Cats were scheduled to be evaluated at three-month intervals, or when signs indicative of a uremic crisis developed. Non-scheduled evaluations were performed at the owner's request. During scheduled visits on months 0, 6, 12, 18 and 24, each cat was evaluated by means of a defined medical history, physical examination, ocular funduscopic examination, body condition score, expanded serum biochemistry panel, serum ionized calcium and parathyroid concentrations, complete blood count, venous blood gas, urinalysis, quantitative urine culture for aerobic bacteria, and indirect blood pressure measurement. On months 3, 9, 15 and 21, the above protocol was followed, except that venous blood gas analysis, serum ionized calcium and serum parathyroid hormone concentrations were not evaluated.

To encourage compliance and determine if there were any medical problems when on-site examinations were not scheduled, telephone interviews of clients were performed monthly. The same veterinary technician performed all telephone interviews. Clients were asked to record the amount of food eaten each day including any food in addition to the assigned food.

Blood Acquisition and Assay. Owners were instructed to withhold food for 8 to 12 hours prior to scheduled evaluations. Blood samples were collected via jugular venipuncture during each visit. Serum was harvested within 30 minutes by standard procedures. If serum for biochemistry profiles could not be processed the same day, serum obtained from centrifuged blood was stored at 4° C. and evaluated the following day. During scheduled visits on months 0, 6, 12, 18 and 24, an aliquot of serum from each collection was frozen (−70° C.) and saved for determinations of PTH and ionized calcium concentrations. Additionally, an aliquot of venous blood was collected in a heparinized blood gas syringe and analyzed within 20 minutes for pH and bicarbonate concentration.

Urine Acquisition and Analysis. Urine was collected by cystocentesis. Urinalyses were performed using a refractometer for urine specific gravity determinations, commercial reagent strips for chemical determinations, and standard technique for sediment evaluation. Quantitative urine cultures for aerobic bacteria were performed on all urine samples. Urine protein concentration was determined by Coomassie brilliant blue dye precipitation and spectrophotometry. Urine creatinine concentrations were determined by an autoanalyzer-based kinetic Jaffe reaction. Urine samples for protein and creatinine determination were stored at 4° C. and analyzed within 24 hours of collection.

Blood Pressure Measurement. All blood pressure determinations were performed in a dedicated room that was not used for any other procedures during the study. Systolic blood pressure measurements were obtained by use of an ultrasonic Doppler monitor using standard techniques.

Patient Management. With the exception of food, the protocol used to manage chronic renal failure was the same for all cats. Likewise, the same protocols were used to manage non-uremic events.

Hypokalemia. In the current study, 9 (20%) of the cats were hypokalemic (serum potassium <3.7 mmol/l) on at least one visit. Five of these cats (3 on the renal food, 2 on the maintenance food) were mildly hypokalemic at the time of food assignment. Treatment was withheld and serum potassium concentration was re-evaluated one month after food assignment to determine if the new food would affect the serum potassium concentrations of these cats. The hypokalemia resolved in all five cats at this time. Of the remaining 4 cats, 2 were treated with potassium citrate at a dose of 40 to 75 mg/kg given orally every 12 h and continued as needed to maintain their serum potassium ≧3.7 mmol/l. The other 2 cats developed hypokalemia coincident with malnutrition secondary to advanced neoplasia.

Hypertension. Seven cats (5 on the renal food, 2 on the maintenance food) developed hypertension during the two-year study period. For the purpose of therapeutic intervention in this study, cats were considered to be hypertensive if their systolic blood pressures were >175 mm Hg on 3 successive visits during a 2-week time period, if evidence of hypertensive retinopathy was associated with a systolic blood pressure >175 mm Hg, or if their systolic blood pressures exceeded 200 mm Hg. All cats requiring therapy were treated with amlodipine besylate at an oral dose of 0.625 mg/cat once daily. Response to therapy was determined after 7 to 14 days of therapy. Dosage adjustment was not required in any cat.

Urinary tract infections. During the study, urinary tract infections (UTI) were detected in each of two cats fed the renal food. Initial episodes of UTI were treated for 21 days with an appropriate antimicrobic as determined by antibiotic susceptibility testing. Response to treatment was evaluated by urinalysis and quantitative bacterial urine culture. Re-infections were detected by follow-up urine cultures; they were treated for a minimum of 6 weeks with an appropriate antimicrobic.

Metabolic acidosis. Any cat having a serum TCO₂ concentration <11.0 mmol/l was further evaluated by venous blood gas analysis. The decision to treat metabolic acidosis was based upon blood bicarbonate concentrations. Eleven cats (2 on the renal food, 10 on the maintenance food) with venous blood HCO₃ concentrations <15.0 mmol/l were treated with potassium citrate (40 to 75 mg/kg given orally every 12 h) with the goal of maintaining blood HCO₃ concentration between 15 and 24 mmol/l. Response to therapy was determined by measuring venous blood HCO₃ concentrations 10 to 14 days after initiating therapy.

Hyperphosphatemia. Hyperphosphatemia (serum phosphorus concentration >6.0 mg/dl) was detected in five cats (2 on the renal food, 3 on the maintenance food) during the study. Of these, one cat (on the maintenance food) had an elevated phosphorus concentration at the time of food assignment. Treatment of this cat with oral phosphorus binding agents was withheld to determine if the assigned food would affect the serum phosphorus concentration. One month after food assignment, the serum phosphorus concentration had returned to the normal range. In order to maintain the serum phosphorus concentration <6 mg/dl in the other 4 cats, aluminum hydroxide was given orally (50-90 mg/kg given orally every 12 h).

Diagnosis of Uremic Crisis. A diagnosis of uremia, a primary endpoint of the study, was made by two clinicians unaware of the food history and not involved in patient management. A diagnosis of uremic crisis was established when all three of the following criteria were evident: 1) owner's observation of at least two clinical signs consistent with uremia including signs of depression, lethargy, anorexia, vomiting, uriniferous breath odor, or uremic stomatitis; 2) serum creatinine concentration at least 20% greater than the previously determined value at which the patient was symptom free; and 3) no plausible alternative for these clinical signs as determined by medical history and physical examination, serum chemistry profile, complete blood count, urinalysis, abdominal radiography and indirect blood pressure determinations.

In one cat, an extrarenal cause was unequivocally determined to have precipitated an episode of uremia. The cat had been lost outdoors for several days in winter, without food or water. After correcting dehydration with lactated Ringer's solution given parenterally, the serum creatinine concentration returned to the pre-crisis levels. The cat remained in the study.

Patient Management after Uremic Crisis. Cats that reached the primary endpoint of the study (e.g., uremic crisis) were treated with parenteral fluids and other appropriate medical care but were not re-introduced into the study. However, the commercially available renal food was fed to all cats after development of a uremic crisis, including all cats that had been previously fed the maintenance food. Two cats died within 30 days of onset of a uremic crisis and three cats survived >30 days after a uremic crisis. Because of the type of analysis used in this clinical trial (i.e., intention-to-treat), all surviving cats were evaluated until death or until completion of the study as if belonging to their initially randomly assigned food groups.

Establishing Causes of Morbidity or Mortality. Based on medical history, physical examination, laboratory results, objective criteria defining uremic crisis (see “Diagnosis of uremic crisis” above) and necropsy results when available, the cause of death or uremic crisis was classified as: 1) definitely not renal, 2) possibly renal, 3) probably renal or 4) definitely renal. Patient deaths or uremic crises classified in category 1 or 2 were designated as renal-unrelated deaths. Patient deaths classified in category 3 or 4 were designated as renal-related death or uremic crises, the primary end-point of the study.

Owner consent for necropsy was requested for all cats that died during the study. After completion of the gross post-mortem exam, tissue samples were harvested and stored in 10% formalin. Paraffin embedded tissue was sectioned at 4 μm, stained with hematoxylin and eosin and examined by light microscopy.

Statistical Analysis. Clinical characteristics were compared between the two dietary groups by use of the Mann-Whitney nonparametric test. Since patients were evaluated at regular intervals by the study protocol (although not all patients were measured at each interval), a mixed between-within subjects analysis of variance (ANOVA) was used to determine if there were significant main effects for Time or Food, and whether the interaction between the two variables (Time by Food) was significant. This analysis is also referred to as a split-plot ANOVA, and allowed evaluation of dependent response variables using all the data while accounting for the correlation between observations. A repeated measure ANOVA was used to assess mean differences between dietary groups for biochemical, physiologic and quality of life measurements for the 12 and 24 month time intervals because most animals had responses at the 12 and 24 month time periods. The repeated measures analysis appropriately accounts for the correlation between repeated observations. Statistical significance for all data was set at P<0.05.

Kaplan-Meier survival curves with Logrank (Mantel-Cox) were used to compare the rates of development of uremic crises and death in both food groups. In addition, the Cox proportional hazard regression model was used to evaluate the effects of food on the relative risk (RR) of development of uremic crises. The same model was used to estimate effects of covariates (systolic blood pressure, urine protein:creatinine ratio and serum creatinine, phosphorus, total CO₂ concentrations) on the development of uremic crises in the renal food group versus the maintenance food group. Relative risk reduction (RRR) was calculated by computing [1−RR]×100%.

Clinical Characteristics of Patients. Forty-five cats met all eligibility criteria and were accepted for study. The treatment group consisted of 22 cats fed the renal food whereas the control group consisted of 23 cats fed the maintenance food. Fourteen neutered males were in the maintenance food group and 8 were in the renal food group. Nine spayed females were in the maintenance food group and 14 were in the renal food group.

At the time of food assignment, there were no statistical differences in overall clinical (age, weight, body condition score, systolic blood pressure) or hematological characteristics of the two groups, as shown in Table 5a.

TABLE 5a
Mean ± SD Values for Hematologic, Serum and Urinary
Variables at Time of Food Assignment

| | Ref. | Renal | Maintenance | P |
|---|---|---|---|---|
| Hematocrit (%) | 26-42 | 34.2 ± 4.0 | 32.3 ± 4.2 | 0.14 |
| SUN (mg/dl) | 14-33 | 41 ± 11 | 49 ± 10 | 0.009 |
| Creatinine (mg/dl) | 0.6-1.4 | 2.51 ± 0.44 | 2.91 ± 0.77 | 0.10 |
| Blood HCO₃ | 17-26 | 19.72 ± 2.03 | 17.45 ± 3.07 | 0.007 |
| Calcium (mg/dl) | 8.9-11.3 | 10.1 ± 0.59 | 9.9 ± 0.38 | 0.66 |
| Phosphorus (mg/dl) | 3.8-8.2 | 3.8 ± 0.8 | 4.0 ± 0.8 | 0.29 |
| PTH (pmol/l) | 0-4 | 7.0 ± 8.2 | 5.2 ± 4.2 | 0.98 |
| Chloride (mmol/l) | 117-128 | 123.3 ± 2.6 | 124.6 ± 2.8 | 0.15 |
| UP/UC ratio | <0.5 | 0.13 ± 0.17 | 0.17 ± 0.14 | 0.19 |

Mean values for Complete Blood Count (CBC) and serum biochemical analytes were within the University of Minnesota Veterinary Medical Center reference range for each group with the exception of serum urea nitrogen, creatinine, blood HCO₃, blood pH and serum parathyroid hormone concentrations. With two exceptions, there were no significant differences between the two food groups in serum, urine and blood biochemical measurements. Significantly higher blood HCO₃ concentrations were observed in the renal food group compared to the maintenance food group (P=0.007). However, mean blood HCO₃ concentrations in both groups were within the reference range. Significantly higher serum urea nitrogen concentrations were observed in the maintenance food group compared to the renal food group (P=0.009). However, there was no difference in magnitude of elevated mean serum creatinine concentration in both groups.

Results of ELISA tests for FIV and FeLV were negative for all 45 cats at food assignment. Likewise, results of RIA testing of serum thyroid hormone concentration were within the normal range (2-4 μg/dl) for all 45 cats at food assignment.

Association between Foods and Serum Biochemical Values. Biochemical and clinical (systolic blood pressure, body weight and body condition score) characteristics of the two groups were compared at 3, 6, 9, 12, 15, 18, 21 and 24 months after food assignment and grouped together to derive overall mean values during the intervals up to 12 months and from 12 to 24 months, as shown in Tables 5b and 5c respectively.

TABLE 5b
Mean ± SD Values for Hematologic, Serum and Urinary
Variables after 12 Months

| | Ref. | Renal | Maintenance | P |
|---|---|---|---|---|
| Hematocrit (%) | 26-42 | 34.9 ± 3.9 | 33.2 ± 4.6 | 0.17 |
| SUN (mg/dl) | 14-33 | 37 ± 9 | 51 ± 13 | <0.0001 |
| Creatinine | 0.6-1.4 | 2.60 ± 0.45 | 2.97 ± 0.80 | 0.076 |
| (mg/dl) | | | | |
| Blood HCO₃ | 17-26 | 19.74 ± 2.40 | 17.14 ± 2.87 | 0.08 |
| Calcium (mg/dl) | 8.9-11.3 | 10.2 ± 0.8 | 10.0 ± 0.5 | 0.29 |
| Phosphorus | 3.8-8.2 | 3.7 ± 0.7 | 4.2 ± 0.8 | 0.05 |
| (mg/dl) | | | | |
| PTH (pmol/l) | 0-4 | 6.1 ± 7.8 | 4.8 ± 3.6 | 0.73 |
| Chloride (mmol/l) | 117-128 | 122.5 ± 2.2 | 124.4 ± 2.4 | 0.0008 |
| UP/UC ratio | <0.5 | 0.18 ± 0.30 | 0.20 ± 0.19 | 0.26 |

TABLE 5c
Mean ± SD Values for Hematologic, Serum and Urinary
Variables After 24 Months

| | Ref. | Renal | Maintenance | P |
|---|---|---|---|---|
| Hematocrit (%) | 26-42 | 34.7 ± 4.06 | 33.5 ± 4.44 | 0.28 |
| SUN (mg/dl) | 14-33 | 39 ± 10 | 51 ± 13 | 0.0007 |
| Creatinine | 0.6-1.4 | 2.68 ± 0.55 | 2.91 ± 0.79 | 0.24 |
| (mg/dl) | | | | |
| Blood HCO₃ | 17-26 | 19.64 ± 2.27 | 17.10 ± 2.64 | 0.02 |
| Calcium (mg/dl) | 8.9-11.3 | 10.4 ± 1.0 | 10.0 ± 0.5 | 0.26 |
| Phosphorus | 3.8-8.2 | 3.9 ± 0.7 | 4.2 ± 0.8 | 0.15 |
| (mg/dl) | | | | |
| PTH (pmol/l) | 0-4 | 5.3 ± 6.9 | 5.2 ± 3.5 | 0.58 |
| Chloride (mmol/l) | 117-128 | 122.2 ± 2.5 | 124.1 ± 2.6 | 0.003 |
| UP/UC ratio | <0.5 | 0.21 ± 0.30 | 0.22 ± 0.25 | 0.48 |

Serum urea nitrogen concentrations were significantly lower and blood bicarbonate concentrations were significantly higher in the renal food group during the 12 and 24 month intervals. Serum chloride and total protein concentrations were significantly lower in the renal food group during the 12 and 24 month intervals, but remained within the laboratory reference range for each group. Serum phosphorus concentration was significantly lower in the renal food group during the 12 month interval, but was not different between the two groups during the 24 month interval. Significant differences were not detected in serum creatinine, albumin, cholesterol, sodium, potassium, calcium, ionized calcium, or PTH concentrations. Likewise, there were no differences between systolic blood pressure, body weight or urine protein creatinine ratios.

Association between Foods and Uremic Crisis. At the conclusion of the study, none of the cats in the renal food group had developed uremic crises, compared with 22% (5) of the cats in the maintenance food group (Table 6). Feeding the renal food was associated with a RRR of 99.9%, compared to feeding the maintenance food. Adjustments for influence of covariates (blood pressure, serum creatinine concentration) on development of uremic crises revealed that the RR in the renal food group remained significantly reduced compared with the maintenance food group.

TABLE 6
Proportions and Hazard Ratio of Uremic Crisis or Death of Cats

| | Renal | | | | |
|---|---|---|---|---|---|
| | Food | Maintenance | | | |
| Events | (%) | Food (%) | HR | 95% CI | P |
| Uremic crises | 0/22 (0) | 5/23 (22) | 0.00 | 0.02-0.69 | 0.02 |
| Renal mortality | 0/22 (0) | 4/23 (17) | 0.00 | 0.02-0.82 | 0.03 |
| All causes mortality | 3/22 (14) | 9/23 (39) | 0.30 | 0.11-1.04 | 0.06 |

CI = Confidence interval;
HR = Hazard ratio

Of the five cats having uremic crises, two responded poorly to therapy and were euthanized within days of the onset of crisis, at the request of the owners. The remaining three cats were euthanized 10, 11 and 20 months post-crisis.

Association between Foods and Death. Kaplan-Meier analysis revealed a significant difference between the renal food and maintenance food groups in renal-related mortality and mortality from all causes (Table 6). When influence of foods on mortality rate related to kidney disease was evaluated, a RRR of 99.9% was detected in the renal food group, compared with the maintenance food group. At the conclusion of the study, 17.4% (4) of the cats in the maintenance food group died from renal causes; there were no renal deaths in the renal food group. Non-renal causes of death or euthanasia in the renal food group included feline infectious peritonitis (1), hit by car (1) and renal carcinosarcoma (1). In the maintenance food group, causes of non-renal death included splenic round cell neoplasia/pancytopenia (1), systemic mast cell tumor (1), lymphosarcoma (2) and not determined (1). Non-renal causes of death did not differ significantly between the renal food (13.6%) and the maintenance food group (21.7%).

When the influence of food on death from all causes was evaluated by use of the Cox proportional hazards model, a RRR of 45.0% was detected in the renal food group as compared to the maintenance food group. By the conclusion of the study, 13.6% (3) of the cats in the renal food group died. During the same interval, 39.1% (9) of the cats assigned to the maintenance food group died.

Necropsy results were available for 8 (renal food 1, maintenance food 7) of the 12 (renal food 3, maintenance food 9) cats that died during the two-year study period. One cat fed renal food for which necropsy results are available was euthanized due to a metastatic renal carcinosarcoma which obliterated most of the renal architecture. Of the 7 necropsies available for cats fed maintenance food, 3 were from cats that had developed a uremic crisis. In all three cases, microscopic evaluation of the kidneys revealed marked lymphoplasmacytic nephritis with marked interstitial fibrosis. Similar lesions were found in the kidneys of the other four cats fed maintenance food for which necropsy reports were available. All four of these cats were euthanized due to advanced neoplasia.

Progression of Chronic Kidney Disease. Serum creatinine concentrations were not significantly different at the beginning of the study, and the magnitude of decline in reciprocal of serum creatinine concentration in the maintenance food group did not differ significantly from that of the renal food group.

Discussion. Results of the current trial support the conclusion that feeding a food specifically designed for treatment of renal failure (renal food) is superior to an adult maintenance food (maintenance food) in minimizing uremic episodes and mortality in cats with non-proteinuric, non-hypertensive, azotemic, spontaneous CRF. In 23 cats fed the maintenance food there were 5 uremic episodes (21.7%) and 4 renal-related deaths (17.4%), whereas no uremic episodes or renal-related deaths occurred in 22 cats fed the renal food. The beneficial effects of the renal food observed in our study are similar to those reported in another dietary clinical trial of cats with spontaneous CRF. In the previously reported study, renal-related mortality in 29 cats fed a food restricted in protein and phosphorus was approximately 33% compared to 52% mortality in 21 cats fed an unrestricted food.

Criteria for timing of dietary intervention in cats with spontaneous CRF have previously been based on empirical observations. Currently there is little debate that when clinical signs of uremia accompany azotemia, dietary modifications such as protein and phosphorus restriction are beneficial. However, a general consensus of opinion does not exist as to when dietary intervention is warranted in cats with clinically stable CRF.

Results of this study indicate that a renal food was beneficial in the management of early feline CRF (serum creatinine concentration >2.0 mg/dl). The data presented here support dietary intervention when the serum creatinine concentration exceeds 2.0 mg/di.

A slowly progressive decline in glomerular filtration rate (GFR) indicated by a gradual increase in serum creatinine concentration was not a characteristic finding in the cats of this study. In the 5 cats that developed uremic crises, abrupt increases (43-371%) in serum creatinine concentrations were preceded by 3 to 21 month periods of stable serum creatinine concentrations. In all 5 cats, abrupt increases in serum creatinine concentrations coincided with appearance of clinical signs typical of uremia (i.e., vomiting, anorexia, dehydration and depression). Retrospective analysis of the results obtained during scheduled tri-monthly evaluations of the history, physical examination, CBC, urinalysis and serum biochemical profile of the 5 cats that developed uremic crises revealed no differences from results obtained during tri-monthly evaluations of 40 cats that did not develop uremic crises. Since the results of our study indicate that the renal food prevented or delayed uremic crises and death, it is recommended that renal foods be initiated for all cats with mild (serum creatinine concentration >2.0 mg/dl) CRF.

With the exception of the 5 cats in the maintenance food group that developed uremic crises, significant differences in the serum creatinine concentrations of cats fed renal food or maintenance food were not observed during the 18 to 24 months they were included in the study. Since 18 cats fed the maintenance food did not develop uremic crises during the 18 to 24 month period of study, an alternative choice would be to initiate therapy with the renal food after the onset of uremic crises. However, only 3 of 5 cats in the maintenance food group that developed uremic crises survived. In addition, sustained increases in serum creatinine concentration followed the onset or uremic crises. Furthermore, after these three cats were withdrawn from the study, long term supportive therapy with oral phosphate binders, potassium citrate, parenteral fluids and a manufactured renal food was required to minimize fluid, electrolyte and acid-base deficits and excesses. These observations support a recommendation of initiating therapy of CRF with a renal food prior to the onset of uremic crisis. Results of the study indicate that withholding the renal food from CRF cats until after the development of a uremic crisis is analogous to adding motor oil to an automobile after the quantity of oil has been depleted to the point that poor lubrication results in irreversible damage to the engine.

Serum creatinine concentration is commonly used as a crude index of GFR. Although it has been suggested that a single serum creatinine measurement may not provide an accurate assessment of renal function, serially performed measurements in the same animal are useful for establishing trends in GFR. Lack of detectable differences in serum concentration of creatinine in renal food and maintenance food cats prior to onset of uremic crises should not be interpreted as irrefutable proof that changes in renal function did not occur. A reciprocal relationship exists between serum creatinine concentration and GFR whereby a 50% reduction in GFR results in a 100% increase in serum creatinine concentration. Serum creatinine concentration is a particularly insensitive indicator of reductions in GFR early in CRF when large changes in GFR are associated small changes in serum creatinine concentration. For example, a 50% reduction in GFR results in an increase in serum creatinine concentration from 1.0 mg/dl to only 2.0 mg/di. A further 50% reduction in GFR would result in an increase in serum creatinine concentration from 2.0 to 4.0 mg/dl. It is possible that differences in renal function between the two food groups in this study may have been detected if inulin clearance studies were used to evaluate GFR. However, the technical difficulty, time and volume of serum required to perform inulin clearance studies on client owned cats precluded their use in this investigation.

At the time of food assignment, significantly higher blood bicarbonate concentrations were observed in the renal food group (mean=19.72±2.03 mmol/l) compared to the maintenance food group (mean=17.45±3.07 mmol/l). Acidosis has been incriminated as a contributing factor in the pathophysiology of uremia in cats, dogs, and humans. However, in this study, mean blood bicarbonate concentrations in both groups were within the University of Minnesota Veterinary Medical Center reference range. The difference between the mean blood bicarbonate concentrations of the two groups remained significant at baseline even when the 5 cats that subsequently developed uremic crises were removed from the analysis.

Summary of Example. A double-masked, controlled, randomized clinical trial was designed to determine if a renal food (modified in protein, phosphorus, sodium and lipid composition) was superior to an adult maintenance food in minimizing uremic episodes and mortality rate in cats with mild to moderate chronic renal failure.

Forty-five client-owned cats were randomly assigned to a maintenance food or a renal food and evaluated tri-monthly for up to 24 months. Kaplan-Meier survival analyses were used to evaluate efficacy of the renal food compared to the maintenance food in minimizing uremia, renal-related mortality, and all-causes of mortality.

| | Renal Food | Maintenance | |
|---|---|---|---|
| Events | (%) | Food (%) | P |
| Uremic crises | 0/22 (0) | 5/23 (22) | 0.02 |
| Renal mortality | 0/22 (0) | 4/23 (17) | 0.03 |
| All causes mortality | 3/22 (14) | 9/23 (39) | 0.06 |

Serum urea nitrogen concentrations were significantly lower and blood bicarbonate concentrations were significantly higher in the group fed the renal food at baseline and during the 12- and 24-month intervals. Cats fed the maintenance food had a significantly greater number of uremic episodes (22%) compared to cats fed the renal food (0%). A significant reduction in renal-related mortality was observed in cats fed the renal food.

The renal food evaluated in this study was superior to an adult maintenance food in minimizing uremic episodes and mortality rate in cats with mild to moderate spontaneous chronic renal failure. A food having reduced levels of protein, phosphorus, and sodium when compared to a standard feline maintenance food is therefore useful for prolonging feline life and delaying the onset of feline renal failure.

In the specification, there have been disclosed typical preferred embodiments of the invention and, although specific terms are employed, they are used in a generic and descriptive sense only and not for purposes of limitation, the scope of the invention being set forth in the following claims. Obviously many modifications and variations of the present invention are possible in light of the above teachings. It is therefore to be understood that within the scope of the appended claims the invention may be practiced otherwise than as specifically described.

What is claimed is:

1. A method for prolonging feline life comprising maintaining a feline on a food having reduced levels of protein, phosphorus, and sodium when compared to a standard feline maintenance food.

2. The method of claim 1 wherein the protein, phosphorus, and sodium are each reduced by at least about 10%.

3. The method of claim 1 wherein the food comprises from about 18% to about 40% protein, from about 0.2% to about 0.85% phosphorus, and from about 0.04% to about 0.35% sodium, on a dry matter basis.

4. The method of claim 1 wherein the food provides from about 3.6 to about 7.9 g/100 kcal ME protein, from about 0.04 to about 0.17 g/100 kcal ME phosphorus, and from about 0.008 to about 0.07 g/100 kcal ME sodium.

5. The method of claim 1 wherein the food comprises a dry food comprising protein in an amount of from about 5% to about 40%, phosphorus in an amount of from about 0.01% to about 2%, and sodium in an amount of from about 0.01% to about 2%, on an “as fed” basis.

6. The method of claim 1 wherein the food comprises a moist food comprising protein in an amount of from about 4% to about 12%, phosphorus in an amount of from about 0.03% to about 0.2%, and from sodium in an amount of from about 0.03% to about 0.2%, on an “as fed” basis.

7. The method of claim 1 wherein the food is supplemented with one or more omega-3 fatty acids.

8. The method of claim 1 wherein the feline is maintained on the food for a period of at least about 6 months.

9. The method of claim 1 wherein the feline is maintained on the food for a period beginning after onset or initial diagnosis of renal disease and continuing for substantially the remainder of the life of the feline.

10. The method of claim 9 further comprising administering to the feline one or more renal drugs.

11. The method of claim 10 wherein the at least one renal drug is selected from the group consisting of ACE inhibitors, endothelin inhibitors, vasopeptide inhibitors, calcium channel blockers, H2 receptor antagonists, proton pump inhibitors, cytoprotectives, antiemetics, androgens, erythropoietin, phosphate binders, calcitriol, and combinations thereof.

12. The method of claim 9 further comprising conducting a periodic diagnostic assessment for the presence or progress of renal disease and adjusting the food and renal drug dosage or regimen based upon the results of the diagnostic assessment.

13. The method of claim 12 wherein the diagnostic assessment is based on one or more criteria selected from the group consisting of blood markers, urine markers, physical observations and measurements, imaging, SNPs, genetic profiles, proteomic profiles, and metabolic profiles.

14. The method of claim 12 wherein said diagnostic assessment is based at least in part on one or more blood markers selected from the group consisting of elevated blood urea nitrogen concentration, elevated serum creatinine concentration, hyperphosphatemia, hyperkalemia, hypokalemia, metabolic acidosis, and hypoalbuminemia.

15. The method of claim 12 wherein said diagnostic assessment is based at least in part on one or more urine markers selected from the group consisting of impaired urine concentrating ability, proteinuria, cylinduria, renal hematuria, inappropriate urine pH, inappropriate urine glucose concentration, and cystinuria.

16. A method for delaying the onset of feline renal failure comprising maintaining a feline on a food having reduced levels of protein, phosphorus, and sodium when compared to a standard feline maintenance food

17. The method of claim 16 wherein each of protein, phosphorus, and sodium are reduced by at least about 10%.

18. A kit useful for prolonging feline life, delaying the onset of feline renal failure, and decreasing the morbidity and mortality caused by feline renal disease comprising in separate containers in a single package a food composition suitable maintaining a feline on a food having reduced levels of protein, phosphorus, and sodium when compared to a standard feline food and at least one of (1) one or more renal drugs and (2) one or more renal diagnostic devices.

19. The kit of claim 18 wherein the food is selected from the group consisting of a dry food comprising protein in an amount of from about 5% to about 40%, phosphorus in an amount of from about 0.01% to about 2%, and sodium in an amount of from about 0.01% to about 2% on an “as fed” basis and a moist food comprising protein in an amount of from about 4% to about 12%, phosphorus in an amount of from about 0.03% to about 0.2%, and from sodium in an amount of from about 0.03% to about 0.2%, on an “as fed” basis.

20. A method for decreasing the morbidity and mortality caused by feline renal disease comprising maintaining a feline on a food having reduced levels of protein, phosphorus, and sodium when compared to a standard feline maintenance food.

21. The method of claim 20 wherein each of protein, phosphorus, and sodium are reduced by at least about 10%.

22. A means for communicating information about or instructions for using a food having reduced levels of protein, phosphorus, and sodium when compared to a standard feline maintenance food to prolong feline life, delay the onset of feline renal failure, and decrease the morbidity and mortality caused by feline renal disease comprising a document, digital storage media, optical storage media, audio presentation, or visual display containing the information or instructions.

23. The means for communicating of claim 22 selected from the group consisting of a label, a brochure, an advertisement, a package insert, a computer-readable medium, an audio presentation, a visual presentation, website pages, and combinations thereof.

24. The means of claim 22 further comprising at least one of information about or instructions for using the food in combination with renal drugs to prevent or treat kidney disease and information about or instructions for using the renal diagnostic devices for determining kidney function and evaluating the presence and severity of kidney disease in a feline.
